(19) United States
(12) Patent Application Publication
(10) Pub. No.: US 2002/0001569 A1
(43) Pub. Date: Jan. 3, 2002

(54) Toothpaste composition with abrasive calcium-based particles having a coating comprising an hydrophobic product

(76) Inventors: Adrien Dromard, Lyon (FR); Sylvie Lavault, Villeurbanne (FR)

Correspondence Address: RHODIA INC., CN-7500, 259 Prospect Plains Road, CHANBURY, NJ, 08512, US

(21) Appl. No.: 09/909,059

(22) Filed: Jul. 19, 2001

Related U.S. Application Data
(63) Continuation of application No. 09/433,246, filed on Nov. 4, 1999.

(30) Foreign Application Priority Data
Nov. 9, 1998 (FR) 98 14231

(51) Int. Cl. A61K 7/18

(52) U.S. Cl. 424/52

(57) ABSTRACT

Abrasive particles which can be used particularly as an abrasive agent in toothpaste compositions and which comprises a core of abrasive calcium-containing material, e.g. a precipitated calcium carbonate, which is coated with a fluorine-compatibilizing hydrophobic coating, e.g. sodium stearate, are provided. Methods of obtaining these abrasive particles, as well as their use in toothpaste compositions are also provided. In these abrasive particles, the compatibility between the abrasive agent and the fluorinated agent is significantly improved.

### FIELD OF THE INVENTION

The field of the present invention is that of toothpaste compositions and, more specifically, of the formulation of the toothpaste compositions with the aid of the various active ingredients in the cleaning and the whitening of teeth, as well as in the fight against caries. The interest lies more particularly, within the context of the invention, in abrasive agents and in fluorinated agents.

### BACKGROUND OF THE INVENTION

Classically, toothpastes comprise one or more abrasive materials which are intended to intervene in the mechanical cleaning of the teeth. These abrasive materials are powders of insoluble particles of defined hardness and shape. Calcium-containing materials such as calcium carbonate and dicalcium phosphate are also commonly used in toothpaste compositions as abrasive agents which participate in the mechanical removal of dental plaque during brushing of the teeth. Calcium carbonate (CC) of natural origin (chalk) or of synthetic origin (precipitated calcium carbonate —PCC—) is one of the oldest dental abrasives; Apart from these calcium-containing abrasives, silica is also largely used in toothpastes as an abrasive, even as a thickener.

It is well known that fluorine as long as it is in the form of fluoride, can chemically bind, in the form of fluoride, with the enamel (or hydroxy-apatite :Ca₅(PO₄)₃OH) in order to convert it into fluoroapatite. This fluorinated enamel is thus much more resistant to attack of the dental plaque, which intervenes, notably, by dissolution in acid media. The result is a better protection from caries. In toothpastes, fluoride-providing products are generally sodium monofluorophosphate (MFP) and sodium fluoride (NaF). The presence of fluorides poses the problem of their compatibility with calcium-based abrasives (calcium carbonate for example), which, due to their surface properties and their aptitude to trap fluorides in the form of insoluble complexes, deprives the toothpaste of its fluorinated ingredient in an active form, from which a loss in therapeutic effectiveness vis-à-vis caries.

This recurrent problem of calcium-containing abrasive/fluorine compatibility in toothpastes has led to a solution outline described in French patent application No. 2,747,569 in the name of the Applicant and relates to a toothpaste composition comprising an abrasive or a silica-based additive and calcium carbonate, which is compatible with fluorine. The silica-based abrasive is here constituted by particles which comprise a coating of dense active silica and a core of calcium carbonate. This coating optionally contains, in an absorbed state, a Ca²⁺ ion stabilising agent (alkali metal phosphate). This technical proposal is interesting but the method of depositing a silica coating onto calcium carbonate crystals is relatively sophisticated. Finally, toothpaste manufacturers would certainly take an abrasive ingredient that was even more economical.

Faced with such a problem, and in the state of the art, the Applicant has fixed, as essential objective, to develop a pulverulent calcium-containing abrasive which has an increased compatibility with respect to fluorine and more specifically a compatibility≧90%, in the case in which the fluorinated agent is sodium fluoride (NaF) or sodium monofluorophosphate (MFP).

Another essential objective of the invention is to provide a toothpaste composition comprising abrasive particles which are based on solid calcium-containing materials, such as CaCO₃ or dicalcium phosphate, and which have a high compatibility with fluorine without this being detrimental to the abrasive power.

Another essential objective of the invention is to provide a toothpaste composition comprising abrasive particles of calcium-containing nature which are compatible with fluorine and which should be economical and simple to obtain.

Another essential objective of the present invention is to provide abrasive particles which can be used in toothpaste compositions, these particles having to be highly compatible with fluorine, economical and simple to obtain.

Another essential objective of the present invention is to provide an inexpensive and easy method to implement for obtaining the above-mentioned abrasive particles.

Another essential objective of the invention is to propose a use of the above-mentioned particles in fluorinated toothpaste formulations with which the action of the fluorine is not hindered by the abrasive.

### SUMMARY OF THE INVENTION

The present invention relates to toothpaste compositions which comprise at least one abrasive agent which is formed from abrasive particles which are constituted by a calcium-based solid material and at least one fluorinated agent, the compatibility between the abrasive agent and the fluorinated agent being significantly improved.

Another objective of the invention is abrasive particles which can be used particularly as an abrasive agent in toothpaste compositions and which comprises a core of abrasive calcium-containing material which is coated with a fluorine-compatibilising hydrophobic coating.

The method of obtaining these abrasive particles, as well as their use in toothpaste compositions, constitute other aspects of the present invention.

After long and laborious research and experimentation, the Applicant has arrived at attaining, in an entirely surprising and unexpected way, all the objectives set forth above, inter alia, by proposing to prevent the contact between the Ca²⁺ ions of the crystalline particles of calcium-containing material on the one hand and, on the other, the fluoride ions which might be present in the toothpaste compositions, by preparing a coating (surface treatment) of said particles with the aid of a hydrophobic product, preferably an organic hydrophobic product.

From where it ensues that the invention relates firstly to a toothpaste composition comprising abrasive particles which comprise a calcium-based solid material and at least one fluorinated agent,

characterised in that the abrasive particles are formed from a calcium-containing material-based core which is coated with a coating which comprises at least one hydrophobic product comprising at least one fatty chain, preferably a C₈-C₂₄ fatty chain.

By thus providing a hydrophobic barrier disposed at the surface of the calcium-containing abrasive particles, the Applicant has noted a very significant improvement in the compatibility of said coated particles with respect to the fluorine.

### DETAILED DESCRIPTION OF THE INVENTION

Advantageously, the hydrophobic product is selected from the group comprising:

fatty acids and salts of fatty acid and of counterion(s) which on the one hand belong to the group comprising ions originating from alkali metals, alkaline earth metals excluding calcium, an ammonium ion, and mixtures thereof, and, on the other hand have a lower affinity for the carboxylic anion than calcium;

fatty alcohols;

or fatty esters, preferably:

natural triglycerides and more preferably still those contained in coconut oil, soya oil, linseed oil, palm oil, sunflower oil or cotton seed oil;

esters of glycerol and of fatty acids;

mono- and di-acetylated esters of glycerol and of fatty acids;

semi-synthetic glycerides;

sucroglycerides;

fatty acid sucroesters;

and mixtures thereof;

the sub-group comprising fatty acids and salts thereof being more particularly preferred.

The fatty acids and more particularly the salts thereof, as defined supra, do in fact have as an additional notable asset, their capacity to trap Ca²⁺ ions and to prevent their diffusion in aqueous media. This effect has the above-mentioned barrier effect.

Preferably, the calcium-based solid material of the abrasive particles is calcium carbonate and/or dicalcium phosphate, it being understood that calcium carbonate is more preferably retained.

Within the sense of the present text, the compatibility of the abrasive particles with respect to the fluorine is understood to be the ratio expressed in % C/Co; C representing the concentration of fluorine ions of a liquid medium which comprises the abrasive particles of calcium-containing nature, and a fluorinated agent (MFP or NAF) after a time t of placing in contact, and Co the initial concentration, at time to, of fluorine ions of the liquid medium considered. The compatibility enables evaluating the trapping of the fluoride ions by the abrasive calcium-containing material.

It is to be noted that the compatibility with respect to the NAF is more severe than that with respect to the MFP. Without wanting to be bound by theory, it is supposed that this is due to the fact that in the MFP, the fluoride ion is integrated in the PO₃F²⁻ ionic entity, such that the calcium ions have probably less affinity with respect to such an entity than with respect to the fluoride ion as it exists in NaF.

It is found that the calcium-containing abrasive particles—preferably CaCO₃— of the toothpaste composition according to the invention have a compatibility with respect to the NaF which can reach 98%. This is extremely indicative of the great effectiveness induced by the invention.

This gain is all the more interesting in that it is obtained without the abrasive properties being found affected.

According to a preferred characteristic of the toothpaste composition of the invention, the coating of the CC particles is based on sodium stearate, potassium stearate or lithium stearate, sodium stearate being more particularly preferred.

According to the invention, the hydrophobic coating is thus formed from an organic product and more particularly from a carboxylic acid salt having a long apolar hydrocarbon chain. These carboxylic acids can comprise one or more carboxylic functions. As the favourable choice of sodium stearate is shown, monocarboxylic acids are preferred. As other examples of fatty acids of which salts, e.g. of alkali metals, can be used within the context of the invention, long chain saturated fatty acids and preferably those the chain length of which is between about 9 and 21 carbon atoms can be cited, such as capric acid, lauric acid, tridecylic acid, myristic acid, palmitic acid or behenic acid. It is possible that said acids be in a salified form, preferably in the form of a sodium salt. It is to be noted that there may be mixtures of fatty acids amongst which unsaturated acids are found, for example oleic acid, but in a minor amount.

In accordance with the invention, it is preferable that the cation of the salt of the acid of the coating have less affinity for the carboxylic anion than calcium, such that the CC or PCC particles or dicalcium phosphate particles attract to themselves the highest number of long chain hydrocarbon carboxylic anions, which thus form an effective hydrophobic barrier between the fluorine on the one hand and the calcium on the other.

In addition to the qualitative aspect, the toothpaste composition according to the invention is quantitatively characterised in that the particles coating ratio, Te, defined by the ratio of

$$\frac{mass of coating}{total mass core + particles coating} \times 100 ,$$

is given below in % dry weight:

0.1≦Te≦30

preferably 0.5≦Te≦40

and more preferably still 1≦Te≦15.

It is clear that the coating ratio Te is preferably adapted to the specific surface area of the particles of calcium-containing material, preferably CaCO₃, to be coated. The larger this specific surface area, the more the coating ratio must consequently be increased in order to ensure the uniformity of the coating.

Furthermore, it is known that the specific surface area of the particles must not go over a certain limit, beyond which limit their size will be too low in order for them to still have abrasive properties. All this problem is therefore to find a compromise between the coating ratio, Te, the specific surface area and the size of the particles of solid calcium-containing material, preferably of CC or PCC.

In accordance with the invention, it has revealed to be advantageous that the abrasive particles of the toothpaste composition be obtained from particles of calcium-containing material, preferably of CC or PCC, the specific surface area BET of which is of the order of 1 to 40 m²/g, preferably of the order of 2 to 15 m²/g. The specific surface area BET is determined according to the method of BRUNAUER-EMET-TELLER described in “The Journal of the American Chemical Society”, Vol. 60, page 309, February 1938 and which corresponds to the standard NFT 45007 (November 1987).

In a preferred embodiment of the invention, according to which the abrasive particles have a core of calcium carbonate, it is convenient to note that this calcium carbonate can be precipitated carbonate (PCC) of the aragonite or calcite type, or even a pulverulent CC natural carbonate obtained by grinding. This PCC or this CC can be of various crystalline forms (for example rhombic, scalenohedric or aragonite). It can be a PCC from the company STURGE: CALOFORT U, CALOPAKE F STURCAL, F, H or L; a PCC from the company SCHAEFER: LIGHT PPC or DENSE PCC; or even a natural CC from the company OMYA: DURCAL 2 or SETACARB OG.

Before coating, the abrasive particles, of calcium-containing material PCC or CC for example, preferably have a median diameter of the order of 0.5 μm to 20 μm. According to an interesting characteristic of the invention, the particle size of the coated abrasive particles, given by D₅₀ in μm, is between 0.5 and 30, preferably between 1 and 20.

The coating of the particles has not a harmful effect upon their abrasive power; on the contrary in fact, the coated abrasive particles have an RDA abrasivity of 20 to 250, preferably of the order of 30 to 200. RDA (“Radioactive Dentine Abrasion”) abrasivity is measured according to the method described by J. J. HEFFERREN in “Journal of Dental Research”, Vol. 55 (4), page 563, 1976.

It has been seen above that the coated abrasive particles in accordance with the invention are particularly compatible with respect to monofluorophosphoric acid MPF and with respect to sodium fluoride NaF. The result is that the fluorinated agent comprised in the toothpaste composition of the invention is preferably a salt of monofluorophosphoric acid MFP and/or an alkali metal fluoride. More preferably still, the fluorinated agent is a sodium, potassium, lithium, calcium, aluminium or ammonium salt of monofluorophosphoric acid, or sodium fluoride.

Furthermore, the inventors have had the credit in demonstrating that it is entirely interesting, with regard to the compatibility of the calcium-containing particles coated with a hydrophobic product (e.g. Na stearate) with respect to fluorine, that the toothpaste composition also comprises at least one surfactant.

Without being limiting, a few details may be given on the quantitative composition of the toothpaste according to the invention by indicating that the toothpaste contains:

preferably of the order of 5 to 40%, and more preferably still of the order of 5 to 35% of its weight, of abrasive particles which comprise a core of calcium-containing material, preferably of calcium carbonate, and a coating of a hydrophobic product, preferably of a fatty acid salt, and more preferably still of Na stearate,

and a fluorinated compound in an amount corresponding to a concentration of the order of 0.005 to 3%, preferably of the order of 0.1 to 1% by weight of fluorine.

Advantageously, the toothpaste composition can further contain:

anionic, non-anionic, amphoteric or zwitterionic surfactant agents, at the rate of about 0.1 to 10%, preferably of about 1 to 5% by weight,

water at the rate of about 5 to 50%, preferably about 10 to 40% by weight,

moisturizing agents, at the rate of about 10 to 85%, preferably 10 to 70% by weight,

thickening agents at the rate of 0.1 to 15% by weight,

and other functional ingredients selected from other polishing abrasives, therapeutic agents, bactericidal agents, anti-microbial agents, anti-plaque agents, flavouring agents, sweetening agents, colouring agents, preservatives.

The following may be cited as examples of surfactants:

C₈-C₁₈ alkyl sulphates which can optionally contain up to 10 oxyethylene and or oxypropylene motifs (sodium lauryl sulphate in particular)

C₈-C₁₈ alkyl sulphoacetates (sodium lauryl sulphate in particular)

C₈-C₁₈ alkyl sulphosuccinates (sodium dioctyl sulphosuccinate in particular)

C₈-C₁₈ alkyl sarcosinates (sodium lauryl sarcosinate in particular)

C₈-C₁₈ alkyl phosphates which can optionally contain up to 10 oxyethylene and or oxypropylene motifs

C₈-C₁₈ carboxylic alkyl ethers containing up to 10 oxyethylene and or oxypropylene motifs

sulphated monoglycerides

non-ionic surfactant agents such as sorbitan fatty esters which are optionally polyethoxylated, ethoxylated fatty acids, polyethyleneglycol esters

amphoteric surfactant agents such as betaines, sulphobetaines.

The moisturisers which can be used in this composition are, for example: glycerol, sorbitol, polyethyleneglycols, lactitol, xylitol.

In practice, the thickening agent can be based on silica such as TIXOSIL 43® marketed by RHODIA or even polymers used alone or in combination such as gum Xanthan, guar gum, cellulose derivatives (Carboxymethylcellulose, hydroxyethylcellulose, hydroxypropylcellulose, hydroxypropylnethylcellulose), cross-linked polyacrylates such as CARBOPOL®s distributed by GOODRICH, alginates or carrageenans, VISCARIN®.

The following may be cited as examples of functional ingredients which can be envisaged for the toothpaste composition of the invention:

other polishing abrasives, such as silica, magnesium carbonate, calcium phosphates, titanium oxides, zinc oxides or tin oxides, talc, kaolin

therapeutic agents, bactericidal agents, anti-microbial agents, anti-plaque agents, such as zinc citrate, polyphosphates, guanidines, bis-guanides or other cationic therapeutic organic compound.

flavouring agents (aniseed essence, star aniseed essence, mint essence, juniper essence, cinnamon essence, clove essence, rose essence), sweetening agents, colouring agents, (chlorophyll), preservatives.

According to a variant, the toothpaste composition can comprise:

Ca²⁺ ion stabilising agents such as hydrosoluble phosphorus derivatives such as alkali metal phosphates (sodium, potassium, or lithium pyrophosphates, orthophosphates, tripolyphosphates, hexametaphosphates).

Finally, the toothpaste composition according to the invention can be presented in various forms, for example as a paste, a gel, or a cream, which are prepared with the aid of conventional methods.

According to another of its aspects, the present invention also relates to abrasive particles usable particularly as an abrasive agent in toothpaste compositions, characterised in that the particles are formed from a core which is based on a solid calcium-containing material, preferably calcium carbonate, and which is coated with a coating which comprises at least one hydrophobic product comprising at least one fatty chain, preferably a C₈-C₂₄ fatty chain.

Advantageously, the hydrophobic product is selected from the group comprising:

fatty acids and salts of fatty acid and of counterion(s) which on the one hand belong to the group comprising ions originating from alkali metals, alkaline earth metals excluding calcium, an ammonium ion, and mixtures thereof, and on the other hand having a lower affinity for the carboxylic anion than calcium;

fatty alcohols;

or fatty esters, preferably:

natural triglycerides and more preferably still those contained in coconut oil, soya oil, linseed oil, palm oil, sunflower oil or cotton seed oil;

esters of glycerol and of fatty acids;

mono- and di-acetylated esters of glycerol and of fatty acids;

semi-synthetic glycerides;

sucroglycerides;

fatty acid sucroesters;

and mixtures thereof;

the sub-group comprising fatty acids and salts thereof being more particularly preferred.

These coated abrasive particles constitute a novel product as such one of the applications of which can be that of abrasive ingredient in toothpaste compositions.

These coated abrasive particles, taken independently from their application in toothpaste, have at least one of the characteristics given above within the context of the description of the toothpaste compositions of the invention. The advantages of these particles and notably their high compatibility with respect to fluorine within the context of toothpaste applications, have largely been set forth above.

As to the method of obtaining these particles, taken on their own or as abrasive ingredient for toothpaste, they can be prepared by implementing the following essential steps:

placing, in a liquid medium, preferably an aqueous medium, a hydrophobic product comprising at least one chain, advantageously a C₈-C₂₄ chain, preferably a carboxylic acid salt

and more particularly still a sodium stearate—in the presence with particles of calcium-containing material—preferably of calcium carbonate-;

in heating and agitating the slurry thus formed so as to enable the coating of said particles;

and then in drying this slurry so as to remove the liquid, to convert this slurry into a powder of particles which are formed from a core of calcium-containing material—preferably of CC or PCC and coated with a hydrophobic product-based coating—preferably Na stearate-based-

A first implementation mode of this method consists essentially:

in preparing a solution/suspension of hydrophobic product, preferably of carboxylic acid salt, and more preferably still of sodium stearate,

in keeping this solution/suspension under agitation at a temperature preferably between 5 and 80° C.;

in incorporating a powder of calcium-containing material, —preferably calcium carbonate—in this solution/suspension;

in heating the slurry obtained at a temperature preferably between 50 and 100° C. whilst keeping it under agitation;

in drying this slurry so as to obtain a powder of abrasive particles, preferably by ovening it and/or by atomising it.

Coating by placing the calcium-containing material, preferably calcium carbonate, in contact with the hydrophobic product, preferably the carboxylic acid salt, and more preferably still sodium stearate, is carried out for several hours at a temperature which can be of the order of 85° C.

Drying by atomisation can be carried out in a classical manner by using air under pressure heated to a temperature between 100 and 150° C.

Drying by ovening can follow or replace the atomisation, takes place in an oven at a temperature of 100±20° C., preferably under vacuum.

According to a variant of this first implementation mode, the preparation of a solution/suspension of hydrophobic product, preferably of carboxylic acid salt (e.g. sodium stearate), can be done away with by simultaneously mixing the abrasive particles to be coated, the coating, and the liquid medium.

A second mode of implementation of the above-mentioned method, which is applicable in the case in which the hydrophobic product is a carboxylic acid salt, preferably a fatty acid salt, consists essentially:

in precipitating—preferably in the hot—the carboxylic acid salt, —preferably the Na stearate—, onto the particles of calcium-containing material—preferably of calcium carbonate—, from the carboxylic acid and from the corresponding base, —preferably stearic acid and NaOH—, which are incorporated in the form of solutions, in a coating container within which these solutions are placed in the presence of the particles of calcium-containing material, —preferably of CC— or PCC— and are mixed with said particles so as to form a main slurry of the precipitation;

and then in producing the powder of particles—preferably of CC or PCC— coated by drying, advantageously by ovening or atomisation.

In practice, according to this second implementation method, stearic acid and water are placed in the presence of sodium hydroxide, NaOH. This mixture is agitated and is heated at a temperature of the order of 90°±10° C., so as to obtain a solution the titre of which is 10% by weight of sodium stearate. An aqueous slurry of 20% PCC by weight/volume heated to 45° C.±10° C. is then incorporated with this solution. The reaction medium is kept under agitation until a coating of the PCC particles is obtained by the alkylated chains of the stearate which form hydrophobic tails all around said particles.

The fourth aspect of the present invention consists in the use as ingredient, particularly as an abrasive agent in toothpaste compositions, of abrasive particles characterised by a structure comprising a calcium carbonate-based core and by a hydrophobic product-based coating, —preferably of Na stearate—such as defined supra.

In accordance with an advantageous disposal of this use, the coated abrasive particles are employed at the rate of 5 to 40%, preferably about 5 to 40%, preferably about 5 to 35% by weight, with respect to the mass of the toothpaste composition.

It is important to underline that the invention, taken in all its aspects, enables obtaining abrasive particles based on calcium-containing material, preferably CaCO₃, coated with a hydrophobic barrier based on a hydrophobic product having at least one fatty chain—advantageously a C₈-C₂₄ fatty chain—preferably based on a carboxylic acid salt, and more preferably still based on Na stearate. NaF compatibilities of the order of 98% are thus obtained, and this in a simple and economical manner by starting with crystalline CaCO₃ particles of the aragonite type, for a coating ratio of 10%.

The Examples which follow will enable to better understand the invention in all its aspects and to make all its advantages and variants of implementation and use re-emerge.

### EXAMPLES

### Example 1

### Preparation of Abrasive PCC Particles Coated with Na Stearate

1.1 Products Used

(i) PCC

STURCAL H corresponding to Aragonite PCCs marketed by the company STURGE and the physico-chemical characteristics of which are given below.

STURCAL H

| | |
|---|---|
| pH at 5% | 9.9 |
| salts sol (%) | 0.1 |
| packed relative density PRD | 0.6 |
| d50 (μm) | 11.2 |
| aragonite content (%) | 80 |
| Compatibility with NaF (%) | |
| (20% abrasive - 24h/37° C.) | |
| water + NaF | 72 |
| sorb/water (75/25) + NaF | 64 |
| sorb/water (75/25) + NaF | 45 |
| sorb/glyc/water (35/35/30) + Naf | 32 |

(ii) Coating/Coat=Na Stearate

Na stearate PROLABO.

(iii) Demineralised Water

1.2 Method

$$\frac{\begin{matrix}{1}^{st} mode of implementation of the method - \\ variant A\end{matrix}}{Coating ratio Te = 2 \%} \qquad \text{1.2.1}$$

a)—Preparation of a Solution Based on Sodium Stearate:

In a glass 5 liter recipient, is loaded:

5-6.12 g of sodium stearate

2000 g of demineralised water

The whole is placed in solution at 65-70° C. under magnetic agitation.

b)—Treatment of Calcium Carbonate.

To the solution obtained in step a) and kept under magnetic agitation, are added 300g of calcium carbonate.

This dispersion is heated at a temperature of 85° C., under magnetic agitation for 5 hours 30 minutes.

The dispersion is then atomised in a Buchi-type atomiser under the following conditions:

pipe air flow rate: 4001/h

temperature of the dispersion: 60-70° C.

temperature of air inlet: 120-125° C.

temperature of air outlet: 80-90° C.

atomisation time: 5 hours

The recovered powder is dried 27 hours in an oven at 100° C. under industrial vacuum.

292.1 g of carbonate treated with 2% of sodium stearate is finally obtained.

1.2.2 1^{st} Mode of Implementation of the Method—Variant B Coating Ratio Te=2%

a)—Preparation of a Solution Based on Sodium Stearate:

In a 250 ml glass recipient is loaded:

0.82 g of sodium stearate

150 g of demineralised water

The whole is placed in solution at 65-65° C. under magnetic agitation.

b)—Treatment of Calcium Carbonate.

To the solution obtained in step a) and kept under magnetic agitation was added 40g of calcium carbonate.

This dispersion is heated at a temperature of about 75° C., under magnetic agitation for 7 hours.

The treated CaCO₃ is filtered, dried 18 hours in an oven at 100° C. under industrial vacuum (300 mm of Hg)

### Example 2

### Tests of Compatibility of PCC Particles Coated with Na Stearate, with Respect to Fluorine, in Liquid Media.

2.1 Preparation of Liquid Medium.

This medium comprises:

20% of abrasive

2% of a surfactant, sodium lauryl sulphate (SLS), at 30% in water,

78% of a aqueous solution of sorbitol (water/sorb=50/50 or 65/35)

comprising about 625 ppm of fluorine in the form of NaF.

a—Mother Solutions of NaF

A. 1—water/sorbitol (50/50) solution having 625 ppm F

Preparation of the following mixture:

1,428.6 g (±0.1 g) of Neosorb 70/70 from the company ROQUETTE FRERES

571.4 g (±0.1 g) of deionised water

Dispersion on RAYNERI

weighing out of 1.3816 g (±0.005 g) of NaF

introduction of the weighed-out amount in a 1 1 graduated flask and completion of the mixture prepared beforehand

placed under agitation under dissolution.

A.2. Water/Sorbitol (65/35) Solution Having 625 ppm F

Preparation of the following mixture:

1,000.0 g (+0.1 g) of neosorb 70/70

1,000.0 g (+0.1 g) of deionised water.

And then proceed as described before in A.1

b—Preparation of the test Liquid Media

1) 7.00 g (+0.05 g) abrasive

2) 0.70 g (+0.05 g) sol. Sodium lauryl sulphate at 30% in water

3) 27.30 g (±0.05 g) sol. sorbitol-water-NaF prepared as above.

c—Preparation of a Control Medium

0.70 g (±0.05 g) sol. Sodium lauryl sulphate at 30% in water

27.30 g (+0.05 g) sol. sorbitol-water-NaF.

d—Contact Time

in an oven at 37° C., under magnetic agitation

duration: 24 h, 48 h, 4 days, 8 days (or other).

e—Preparation of Samples to be Determined

Take about 1 ml of homogeneous suspension (or of control solution)

Filter over 0.45 μm

Weigh 0.50 g (±0.02 g) of filtrate

Complete to 10.00 g (±0.02 g) with deionised water.

Add 10 ml of TAFIC buffer

f—Standardisation of the Ionometer

f1—“TARE” solution 10 ml solution having 3.8 ppm F^{(*)} 10 ml TAFIC buffer

f2—“STANDARDISATION” solution 10 ml solution having 38 ppm F^{(*)} 10 ml TAFIC buffer

^{(*)} Prepared from commercial 0.01 M NaF solution

g—Compatibility

$$Compatibility ( \% ) = \frac{concentration read for the test}{concentration read for control} \times 100$$

2.2 Results and Conditions of the Tests

a—Water/Sorbitol (50/50) Solution (Tests 1 to 5)

The coating of the PCC. in tests 2 and 3, is carried out according to variant B given supra in point 1.2.2. (filtration-drying), while variant A (atomisation cf. 1.2.1 supra)—is used for tests 4, 5.

The results are given in Table 1 below.

TABLE 1
COMPATIBILITY C in % with respect to the NaF

| N° test | | | | | |
|---|---|---|---|---|---|
| % coating | 1 | 2 | 3 | 4 | 5 |
| duration | 0 | 0.5 | 2 | 6 | 10 |
| −24 h | 49 | 80 | 87 | 91 | 80 |
| −48 h | / | / | / | 85 | 70 |
| −3 d | 39 | 68 | 83 | 85 | 66 |
| −4 d | 37 | 62 | 82 | 81 | 64 |
| −5 d | / | / | / | / | / |
| −6 d | 34 | 54 | 81 | / | / |
| −8 d | 31 | 50 | 80 | 80 | 80 |

b Water/Sorbitol (63/35) Solution (Tests 6 to 8)

The coating of the PCC particles in tests 7 and 8 is done according to variant B (point 1.2.2-supra)—filtration/drying.

Table 2 below gives the results obtained.

TABLE 2
COMPATIBILITY C in % with respect to the NaF

| N° tests | | | |
|---|---|---|---|
| % coating | 6 | 7 | 8 |
| duration | 0 | 2 | 0.5 |
| −24 h | 61 | 89 | 78 |
| −48 h | 54 | 86 | 70 |
| −4 d | 47 | 85 | 62 |
| −6 d | 43 | 86 | 56 |
| −8 d | 39 | 85 | 52 |

### Example 3

### Tests of Compatibility of the PCC Particles Coated with Na Stearate, with Respect to Fluorine, in Toothpaste Compositions.

3.1 Preparation of the Toothpaste Compositions.

Compatibility test/NaF and /MFP ageing at 37° C.—Determinations of stable fluorine

10 g of toothpaste paste of the following composition:

| Composition | % by weight |
|---|---|
| carboxymethylcellulose | 0.8 |
| sorbitol | 18 |
| sodium monofluorophosphate (MFP) | 0.8 or |
| or sodium fluoride (NaF) | 0.24 |
| sodium saccharinate (sweetener) | 0.2 |
| sodium benzoate (preservative) | 0.3 |
| abrasive tested | 40 |
| Tixosil 43 (thickening silica) | 5 |
| sodium lauryl sulphate (30% aqueous solution) | 4 |
| flavour | 0.8 |
| deionised water | qsp 100 |

are placed in suspension in 90 g of water, for 5 minutes at ambient temperature. The suspension is then centrifuged and the supernatant filtered.

The determination by ionometry of the total soluble fluorine in the supernatant necessitates in the case of MFP a prior treatment of hydrolysis in acid medium, which releases the fluorine ions present in the form of a complex in the MFP.

The hydrolysis is carried out in the presence of 25 ml of supernatant with 5 ml of 5N sulphuric acid and 20 ml of deionised water, and placed in an oven at 60° C. for at least 5 hours.

The fluorine concentration of the supernatant is measured after hydrolysis, as well as before hydrolysis.

3.2 Conditions and Results of Tests (9 to 12)

Table 3 below gives the results obtained with respect to NaF (tests 9 and 10). Table 4 gives the results obtained with respect to MFP (tests 11 to 12).

Stearate Coated PCC Samples

Tests in toothpaste formulation with NaF (1044 ppmF) Ageing at 37° C.—Determinations of soluble fluorine F and compatibility C

TABLE 3

| | | |
|---|---|---|
| Test N° | 9 | 10 |
| coating | 0 | 2 |

| Duration | pH | F (ppm) | pH | F (ppm) | C (%) |
|---|---|---|---|---|---|
| −to | 9.45 | 900 | 9.45 | 920 | |
| −24 h | 9.6 | 340 | 9.6 | 690 | 75 |
| −48 h | 9.6 | 310 | 9.6 | 660 | 72 |
| −7 d | 9.8 | <200 | 9.6 | 590 | 64 |
| −14 d | 9.8 | I | 9.6 | 570 | 62 |
| −1 | 9.8 | <50 | 9.8 | 520 | 56.5 |
| month | | | | | |

PASTES OF LOW VISCOSITIES WITH COATED PCC SAMPLES

Coated PCC Samples

Tests in toothpaste formulation with MFP (1024 ppmF) Ageing at 37° C.—Determinations of total fluorine F and compatibility C

TABLE 4

| Test | ABRASIVE (40%) | to/AT*/RT | | | 3 weeks/37° C. | | | 6 weeks/37° C. | | | 9 weeks/37° C. | | | 12 weeks/37° C. | | |
|---|---|---|---|---|---|---|---|---|---|---|---|---|---|---|---|---|
| 11 | PCC | 9.1 | 975 | 100 | 9.0 | 775 | 79 | 9.1 | / | / | 9.05 | 700 | 72 | 9.1 | 685 | 70 |
| | non-coated | | | | | | | | | | | | | | 675 | |
| | | | | | | | | | | | | | | | CP71 | |
| | | | | | | | | | | | | | | | 0 | |
| 12 | PCC | 9.2 | 980 | 100 | 9.1 | 960 | 98 | 9.05 | 900 | 92 | 9.05 | 885 | 90 | 9.05 | 835 | 85 |
| | coated | | | | | | | | | | | | | | | |
| | 2% Na stearate | | | | | | | | | | | | | | | |

*AT: ambient temperature

### Example 4

### Tests of Compatibility C, with Respect to NaF, of Particles of PCC Coated with an Ester of Glycerol and of Fatty Acid and an Ester of Sucrose and of Fatty Acid

The coating is carried out as described in Example 1 for Na stearate, the difference being that the coating is Geleol Sucroester 7 (glycerol monopalmitato-stearate/sucrose distearate).

The Te is 7%.

The conditions of measurement of the compatibility C with respect to NaF are those of Example 2 with a water/sorbitol (50/150) solution having 625 ppm fluorine.

Table 5 above gives the results obtained for tests 13 (control) and 14. Compatibility C in % with respect to the NaF (PCC coated with Geleol sucroester 7).

TABLE 5
COMPATIBILITY C in % with respect to the NaF
PCC coated with Geleol sucroester 7

| N° Test | 13 | 14 |
|---|---|---|
| % coating | | |
| Duration | 0 | 7 |
| 8d | 33 | 47 |

1. A toothpaste composition comprising abrasive particles which comprise a calcium-based solid material and at least one fluorinated agent, wherein the abrasive particles have a calcium-containing material-based core and a calcium-containing material-based coating on said core which comprises at least one hydrophobic product comprising at least one fatty chain.

2. A toothpaste composition according to claim 1 wherein said fatty chain is a C₈-C₂₄ fatty chain.

3. A toothpaste composition according to claim 1 wherein the hydrophobic product is selected from the group consisting of:
fatty acids and salts of fatty acid and of counterion(s) which on the one hand belong to the group comprising ions originating from alkali metals, alkaline earth metals excluding calcium, an ammonium ion, and mixtures thereof, and, on the other hand have a lower affinity for the carboxylic anion than calcium;
fatty alcohols; and
fatty esters.

4. A toothpaste composition according to claim 3 wherein the hydrophobic product is selected from the group consisting of:
natural triglycerides and more preferably still those contained in coconut oil, soya oil, linseed oil, palm oil, sunflower oil or cotton seed oil;
esters of glycerol and of fatty acids;
mono- and di-acetylated esters of glycerol and of fatty acids;
semi-synthetic glycerides;
sucroglycerides;
fatty acid sucroesters; and
mixtures thereof.

5. The toothpaste composition according to claim 1 wherein the calcium-based solid material of the abrasive particles is calcium carbonate and/or dicalcium phosphate.

6. The toothpaste composition according to claim 1 wherein the calcium-based solid material of the abrasive particles is calcium carbonate.

7. The toothpaste composition according to claim 1 wherein said hydrophobic product is based on sodium stearate, potassium stearate or lithium stearate.

8. The toothpaste composition according to claim 1 wherein said hydrophobic product is based on sodium stearate.

9. The toothpaste composition according to claim 1 wherein the coating ratio of the particles, Te, defined by the ratio of
$$\frac{mass of coating}{total mass core + particles coating} \times 100 ,$$
is in % dry weight: 0.1≦Te≦30.

10. The toothpaste composition according to claim 9 wherein 0.5≦Te≦40.

11. The toothpaste composition according to claim 9 wherein 1≦Te≦15.

12. The toothpaste composition according to claim 1 wherein the abrasive particles are obtained from particles of calcium-containing material, the specific surface area BET of which is of the order of 1 to 40 m²/g.

13. The toothpaste composition according to claim 1 wherein the abrasive particles are obtained from particles of calcium-containing material, the specific surface area BET of which is of the order of 2 to 15 m²/g.

14. The toothpaste composition according to claim 1 wherein the particle size of the abrasive particles, given by D₅₀ in μm, is between 0.5 and 30.

15. The toothpaste composition according to claim 1 wherein the particle size of the abrasive particles, given by D₅₀ in μm, is between 1 and 20.

16. The toothpaste composition according to claim 1 wherein the abrasive particles have an RDA abrasivity of the order of 20 to 250.

17. The toothpaste composition according to claim 1 wherein the abrasive particles have an RDA abrasivity of the order of 30 to 200.

18. The toothpaste composition according to claim 1 wherein the fluorinated agent is selected from the group consisting of a salt of monofluorophosphoric acid, an alkali metal fluoride, and mixtures thereof.

19. The toothpaste composition according to claim 18 wherein the fluorinated agent is selected from the group consisting of a sodium, potassium, lithium, calcium, aluminium or ammonium salt of monofluorophosphoric acid, and sodium fluoride.

20. The toothpaste composition according to claim 1 further comprising at least one surfactant.

21. The toothpaste composition according to claim 1 wherein it contains of the order of 5 to 40% of said abrasive particles and a fluorinated agent in an amount corresponding to a concentration of the order of 0.005 to 3% by weight of fluorine.

22. The toothpaste composition according to claim 1 wherein it contains of the order of 5 to 35% of its weight of said abrasive particles and a fluorinated agent in an amount corresponding to a concentration of the order of 0.1 to 1% by weight of fluorine.

23. The toothpaste composition of claim 22 wherein said core is calcium carbonate and said hydrophobic product is Na stearate.

24. The toothpaste composition according to claim 1 wherein it further contains:
anionic, non-anionic, amphoteric or zwitterionic surfactant agents, in an amount of about 0.1 to 10% by weight of said composition,
water in an amount of about 5 to 50% by weight of said composition,
moisturizing agents in an amount of about 10 to 85% by weight of said composition,
thickening agents in an amount of 0.1 to 15% by weight by weight of said composition, and
optional ingredients selected from other polishing abrasives, therapeutic agents, bactericidal agents, anti-microbial agents, anti-plaque agents, flavouring agents, sweetening agents, colouring agents, preservatives.

25. The toothpaste composition according to claim 24 wherein said surfactant agents are present in an amount of about 1 to 5% by weight by weight of said composition, said water is present in an amount of about 10 to 40% by weight by weight of said composition, and said moisturizing agents are present in an amount of about 10% to 70% by weight of said composition.

26. The toothpaste composition according to claim 1 wherein said composition is in the form of a paste, a gel or a cream.

27. Abrasive particles usable particularly as an abrasive agent in toothpaste compositions comprising, a core which is based on a solid calcium-containing material, and a coating on said core which comprises at least one hydrophobic product comprising at least one fatty chain.

28. The particles according to claim 27 wherein said core is calcium carbonate and said fatty chain is a C₈-C₂₄ fatty chain.

29. The particles according to claim 27 wherein the hydrophobic product is selected from the group consisting of:
fatty acids and salts of fatty acid and of counterion(s) which on the one hand belong to the group comprising ions originating from alkali metals, alkaline earth metals excluding calcium, an ammonium ion, and mixtures thereof, and, on the other hand have a lower affinity for the carboxylic anion than calcium;
fatty alcohols; and
or fatty esters.

30. The particles according to claim 27 wherein the hydrophobic product is selected from the group consisting of:
natural triglycerides and more preferably still those contained in coconut oil, soya oil, linseed oil, palm oil, sunflower oil or cotton seed oil;
esters of glycerol and of fatty acids;
mono- and di-acetylated esters of glycerol and of fatty acids;
semi-synthetic glycerides;
sucroglycerides;
fatty acid sucroesters ; and
mixtures thereof.

31. The particles according to claim 27 wherein the calcium-based solid material of the abrasive particles is calcium carbonate and/or dicalcium phosphate.

32. The particles according to claim 27 wherein the calcium-based solid material of the abrasive particles is calcium carbonate.

33. The particles according to claim 27 wherein said hydrophobic product is based on sodium stearate, potassium stearate or lithium stearate.

34. The particles according to claim 27 wherein said hydrophobic product is based on sodium stearate.

35. The particles according to claim 27 wherein the coating ratio of the particles, Te, defined by the ratio of
$$\frac{mass of coating}{total mass core + particles coating} \times 100 ,$$
is in % dry weight: 0.1≦Te≦30.

36. The particles according to claim 35 wherein 0.5≦Te ≦40.

37. The particles according to claim 35 wherein 1≦Te ≦15.

38. The particles according to claim 27 wherein the abrasive particles are obtained from particles of calcium-containing material, the specific surface area BET of which is of the order of 1 to 40 m²/g.

39. The particles according to claim 27 wherein the abrasive particles are obtained from particles of calcium-containing material, the specific surface area BET of which is of the order of 2 to 15 m²/g.

40. The particles according to claim 27 wherein the particle size of the abrasive particles, given by D₅₀ in μm, is between 0.5 and 30.

41. The particles according to claim 27 wherein the particle size of the abrasive particles, given by D₅₀ in μm, is between 1 and 20.

42. The particles according to claim 27 wherein the abrasive particles have an RDA abrasivity of the order of 20 to 250.

43. The particles according to claim 27 wherein the abrasive particles have an RDA abrasivity of the order of 30 to 200.

44. A method of obtaining abrasive particles consisting essentially of:
placing, in a liquid medium, a hydrophobic product comprising at least one fatty chain, in the presence of particles of calcium-containing material;
heating and agitating the slurry thus formed so as to enable the coating of the particles; and
drying said slurry so as to remove the liquid, to convert this slurry into a powder of particles which are formed from a core of calcium-containing material coated with a hydrophobic product-based coating.

45. The method according to claim 44 wherein said liquid medium is an aqueous medium.

46. The method according to claim 44 wherein said fatty chain is a C₈-C₂₄ chain.

47. The method according to claim 44 wherein said hydrophobic product is a carboxylic acid salt.

48. The method according to claim 44 wherein said hydrophobic product is sodium stearate.

49. The method according to claim 44 wherein said core of calcium-containing material is calcium carbonate.

50. The method according to claim 44 wherein said core of calcium-containing material is precipitated calcium carbonate.

51. The method according to claim 44, characterised in that it consists essentially of:
preparing a solution/suspension of hydrophobic product comprising at least one chain,
agitating said solution/suspension,
incorporating a powder of calcium-containing material in said solution/suspension;
in heating the slurry whilst keeping it under agitation; and
in drying this slurry so as to obtain a powder of abrasive particles.

52. The method according to claim 51 wherein said hydrophobic product is a carboxylic acid salt.

53. The method according to claim 51 wherein said hydrophobic product is sodium stearate.

54. The method according to claim 51 further comprisng keeping said solution/suspension at a temperature preferably between 5 and 80° C. during said agitating.

55. The method according to claim 51 wherein said calcium-containing material is calcium carbonate.

56. The method according to claim 51 wherein the temperature of said slurry during said heating is between 50 and 100° C.

57. The method according to claim 51 wherein said drying is by ovening said slurry.

58. The method according to claim 51 wherein said drying is atomising said slurry.

59. A method of obtaining abrasive particles consisting essentially of:
placing a carboxylic acid salt in the form of an aqueous solution thereof in the presence of an aqueous slurry particles of calcium-containing material in a coating container, and mixing so as to form a main slurry of the precipitation; and
producing a powder of abrasive particles by drying said main slurry.

60. The method according to claim 59 wherein said carboxylic acid salt is sodium stearate.

61. The method according to claim 59 wherein said of calcium-containing material is calcium carbonate.

62. The method according to claim 59 wherein said of calcium-containing material is precipitated calcium carbonate.

63. The method according to claim 59 wherein said drying is by ovening said slurry.

64. The method according to claim 59 wherein said drying is atomising said slurry.

65. The method of claim 59 wherein said aqueous solution is a hot solution of said carboxylic acid salt which is prepared from the carboxylic acid and from the corresponding base.
